(19) United States
(12) Patent Application Publication
(10) Pub. No.: US 2015/0216495 A1
(43) Pub. Date: Aug. 6, 2015

(54) ROBOTICALLY ASSISTED SELF-ALIGNING PORTABLE X-RAY MACHINE

(71) Applicant: Gabriel Gonzalez, McAllen, TX (US)

(72) Inventor: Gabriel Gonzalez, McAllen, TX (US)

(21) Appl. No.: 14/612,976

(22) Filed: Feb. 3, 2015

Related U.S. Application Data
(60) Provisional application No. 61/936,211, filed on Feb. 5, 2014.

(51) Int. Cl. A61B 6/00

(52) U.S. Cl. CPC A61B 6/4476; A61B 6/4405

(57) ABSTRACT

A robotically assisted self-aligning portable x-ray machine is disclosed. The machine includes an accelerometer in a digital cassette and an accelerometer in the collimator box. Both accelerators are connected to a micro control unit (MCU), which receives the inclination along at least two axes from both accelerometers. The MCU controls a first stepper motor and a second stepper motor to align the collimator box with the cassette according to an x-ray exam protocol. Inclination of the components is based on the exam protocol. Inclination data is recorded on an x-ray image exposed after alignment. The inclination data may be used by a radiologist or a physician in making a diagnosis based on the x-ray image.

### CLAIM OF BENEFIT TO PRIOR APPLICATION

This application claims benefit to U.S. Provisional Patent Application 61/936,211, entitled “ROBOTICALLY ASSISTED SELF-ALIGNING PORTABLE X-RAY MACHINE,” filed Feb. 5, 2014. The U.S. Provisional Patent Application 61/936,211 is incorporated herein by reference.

### BACKGROUND

The embodiments herein relate generally to a robotically assisted self-aligning portable x-ray machine.

Existing portable x-ray machines have no way of verifying the position of the x-ray cassette and the x-ray tube relative to each other. Existing portable x-ray machines also do not have the ability to self-align the x-ray tube to the cassette for a given x-ray exam protocol.

Currently, a radiologist must rely on the word of an x-ray technologist as to what position the patient was in for a portable x-ray exam that requires the patient to be at a given position. Thus, the radiologist doesn't know for sure if the patient was positioned properly, which may result in an incorrect diagnosis for that patient. Setting the x-ray tube on a portable unit perpendicularly to the cassette or according to a given exam protocol is guess work. If the x-ray beam is not perpendicular to the cassette or set to a certain angle relative to the x-ray cassette, the patient's anatomy can appear distorted. The distortion can result in a misdiagnosis or in repeating the x-ray procedure, which exposes the patient to a greater and unnecessary radiation dose.

### BRIEF DESCRIPTION

According to one embodiment of the present invention, a system robotically self-aligns components of an x-ray machine to produce more accurate images and better diagnoses. The system includes a first accelerometer located in a digital cassette of an x-ray machine. The first accelerometer is configured to determine an inclination of the digital cassette on at least two axes and to transmit the inclination data. A second accelerometer is located in a collimator box of the x-ray machine. The second accelerometer is configured to determine an inclination of the collimator box on at least two axes and to transmit the inclination data. A micro control unit (MCU) is connected to the first accelerometer and to the second accelerometer. The MCU is configured to receive the inclination data from the first accelerometer and from the second accelerometer. The system also includes a first stepper motor and a second stepper motor. The first stepper motor is disposed in a first joint of the x-ray machine. The first stepper motor is connected to the MCU and configured to adjust the angle, along a first axis, of the collimator box relative to the digital cassette based on instructions received from the MCU. The second stepper motor is disposed in a second joint of the x-ray machine. The second stepper motor is connected to the MCU and configured to adjust the angle, along a second axis, of the collimator box relative to the digital cassette based on instructions received from the MCU.

The robotically assisted self-aligning portable x-ray machine is able to self-align according to a selected exam protocol. In some embodiments, when an exam protocol is selected, the robotically assisted self-aligning portable x-ray machine activates either or both of (i) the first stepper motor to adjust the angle, along the first axis, of the collimator box relative to the corresponding angle of the digital cassette and according to the selected exam protocol and (ii) the second stepper motor to adjust the angle, along the second axis, of the collimator box relative to the corresponding angle of the digital cassette and according to the selected exam protocol. When the angles are adjusted and the components are aligned according to the exam protocol, the x-ray exposure is made and inclination data for the digital cassette and the collimator box is recorded on the x-ray image, thereby allowing the inclination data to be used by a radiologist or a physician in making a diagnosis based on the x-ray image.

### BRIEF DESCRIPTION OF THE DRAWINGS

Having described the invention in general terms, reference is now made to the accompanying drawings, which are not necessarily drawn to scale, and wherein:

FIG. 1 conceptually illustrates a side schematic view of a robotically assisted self-aligning portable x-ray machine in some embodiments.

FIG. 2 conceptually illustrates a front schematic view of the x-ray machine of FIG. 1.

FIG. 3 conceptually illustrates a section view of the x-ray machine, taken along line 3-3 in FIG. 2.

FIG. 4 conceptually illustrates a section view of the x-ray machine, taken along line 4-4 in FIG. 2.

FIG. 5 conceptually illustrates a section view of the x-ray machine, taken along line 5-5 in FIG. 1.

FIG. 6 conceptually illustrates a section view of the x-ray machine, taken along line 6-6 in FIG. 1.

FIG. 7 conceptually illustrates a flow chart for performing robotically assisted self-alignment of a portable x-ray machine in some embodiments.

FIG. 8 conceptually illustrates an electrical schematic of the x-ray machine of FIG. 1.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

Some embodiments include a system that produces more accurate images and better diagnoses by way of a robotically assisted self-aligning portable x-ray machine that self-aligns according to a selected exam protocol and that provides an x-ray image with recorded inclination data related to the x-ray exposure made for the procedure, thereby allowing the inclination data to be used by a radiologist or a physician in making a diagnosis based on the x-ray image.

In some embodiments, the system includes a first accelerometer located in a digital cassette of an x-ray machine. The first accelerometer is configured to determine an inclination of the digital cassette on at least two axes and to transmit the inclination data. In some embodiments, the system includes a second accelerometer located in a collimator box of the x-ray machine. The second accelerometer is configured to determine an inclination of the collimator box on at least two axes and to transmit the inclination data. In some embodiments, the system includes a micro control unit (MCU) connected to the first and second accelerometers. The MCU is configured to receive the inclination data from the first accelerometer and from the second accelerometer. In some embodiments, the system further includes a first stepper motor and a second stepper motor. The first stepper motor is disposed in a first joint of the x-ray machine. The first stepper motor is connected to the MCU and configured to adjust the angle, along a first axis, of the collimator box relative to the digital cassette based on instructions received from the MCU. The second stepper motor is disposed in a second joint of the x-ray machine. The second stepper motor is connected to the MCU and configured to adjust the angle, along a second axis, of the collimator box relative to the digital cassette based on instructions received from the MCU.

In some embodiments, the robotically assisted self-aligning portable x-ray machine is able to utilize the system to self-align according to a selected exam protocol. In some embodiments, when an exam protocol is selected, the robotically assisted self-aligning portable x-ray machine activates either or both of (i) the first stepper motor to adjust the angle, along the first axis, of the collimator box relative to the corresponding angle of the digital cassette and according to the selected exam protocol and (ii) the second stepper motor to adjust the angle, along the second axis, of the collimator box relative to the corresponding angle of the digital cassette and according to the selected exam protocol. When the angles are adjusted and the components are aligned according to the exam protocol, the x-ray exposure is made and inclination data for the digital cassette and the collimator box is recorded. The inclination data may, for example, appear in the digital x-ray image, or it may be recorded as metadata in the image file, thereby allowing the inclination data to be used by a radiologist or a physician in making a diagnosis based on the x-ray image. Specifically, the radiologist can give a precise and accurate dictation of the positioning of the patient via the inclination data recorded from the system on the digital x-ray image, thus allowing the radiologist or physician to make a diagnosis that is fully-informed by the inclination data. For diagnostic purposes, this is a great improvement over the current practices of radiologists who use any of the other portable x-ray units for diagnostic analysis because none of the existing portable x-ray units provide the accuracy and certainty in knowing the precise measurements of the inclination data.

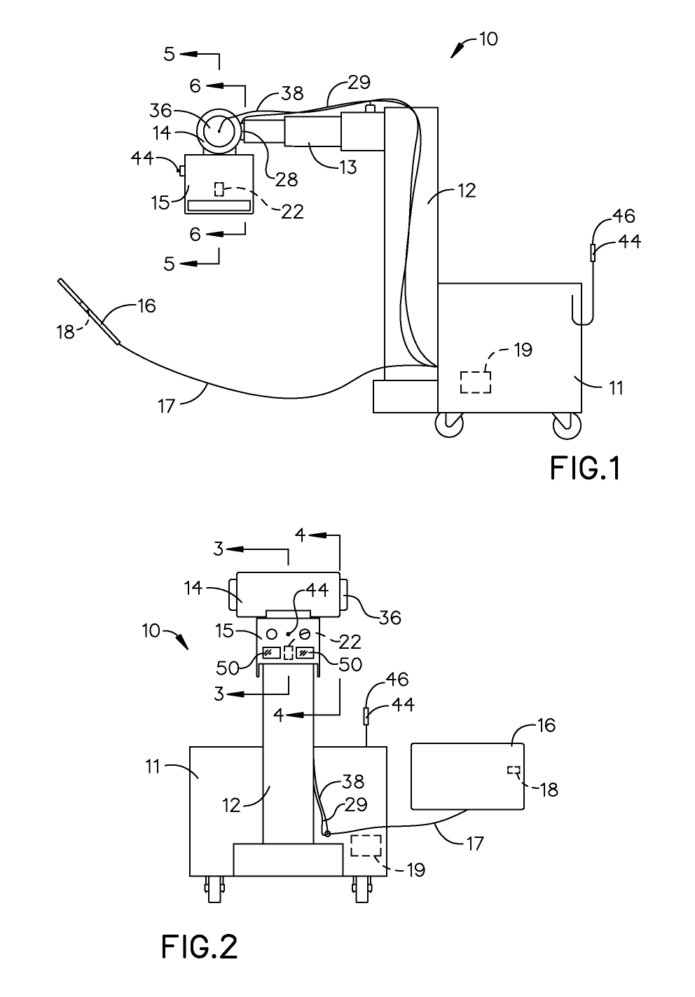

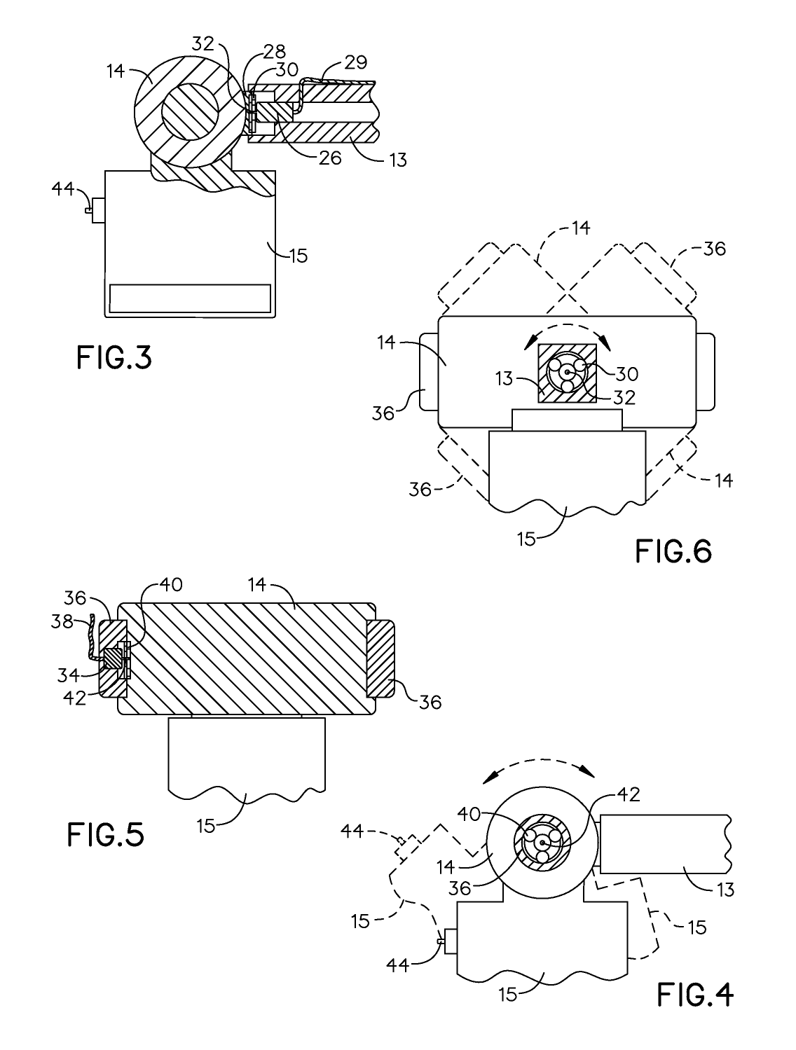

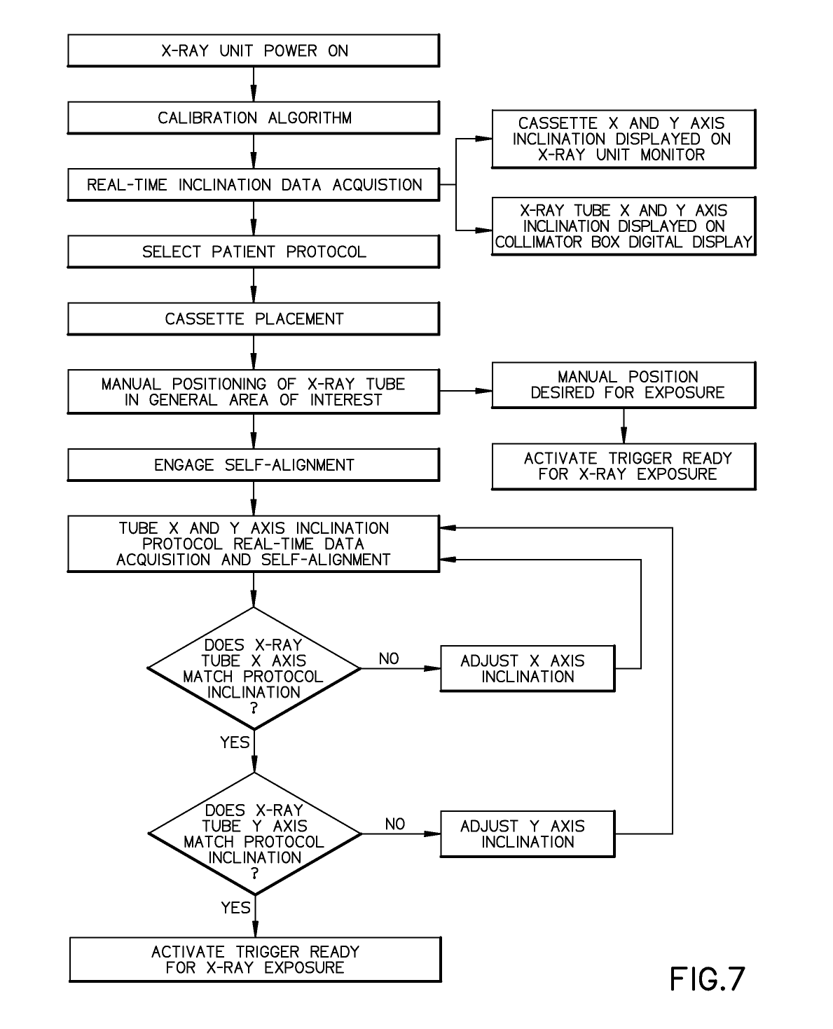

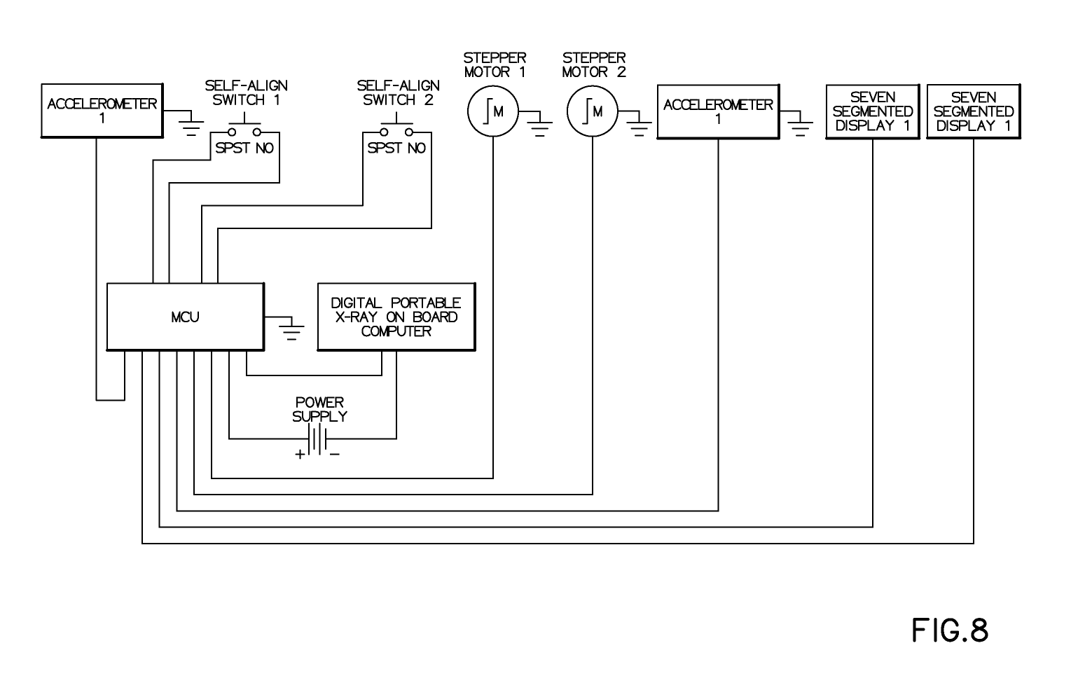

By way of example, and referring to FIGS. 1-8, one embodiment of the present invention comprises a robotically assisted self-aligning portable x-ray machine 10. The x-ray machine 10 may be similar to like devices that are well-known in the art. For example, the x-ray machine 10 may include an x-ray cart 11, which may carry components such as power supply, onboard computer, and so on. A vertical arm 12 may be connected to the front of the cart 11, and a telescoping arm 13 may extend from the vertical arm 12. A pivoting arm 14 may be connected to the end of the telescoping arm 13, and a collimator box 15 may be connected to the pivoting arm 14. The collimator box 15 may contain an x-ray tube or other apparatus for emitting x-rays. The pivoting arm 14 may provide the collimator box 15 with independent rotation on both an X-axis and a Y-axis. Greater or fewer axes may be used. A digital cassette 16 may sense or receive the x-rays from the collimator box 15 and produce a digital image. The digital cassette 16 may be connected to the x-ray machine 10 by a power and communication cable 17.

A multi-axis accelerometer 18 may be located within the digital cassette 16, and the cassette accelerometer 18 may be connected to a micro control unit (MCU) 19 with power and communication cable 17. A second multi-axis accelerometer 22 may be located within the collimator box 15. The collimator accelerometer 22 may be connected to the MCU 19. The MCU 19 may be connected to portable x-ray machine's onboard computer and/or power supply (not shown).

The x-ray machine 10 may include an X-axis stepper motor 26 located in the X-axis joint assembly 28 of the x-ray machine 10. The X-axis stepper motor 26 may be connected to the MCU 19 by a power and communication cable 29. An X-axis planetary gear system 30 may be located in the X-axis joint assembly 28 of the x-ray machine 10. The X-axis stepper motor 26 may be connected to a center gear of the X-axis planetary gear system 30 with an X-axis drive shaft 32. The machine 10 may include an Y-axis stepper motor 34 located in the Y-axis joint assembly 36 of the x-ray machine 10. The Y-axis stepper motor 34 may be connected to the MCU 19 by a power and communication cable 38. A Y-axis planetary gear system 40 may be located in the Y-axis joint assembly 36 of the x-ray machine 10. The Y-axis stepper motor 34 may be connected to a center gear of the Y-axis planetary gear system 40 with a Y-axis drive shaft 42.

The machine 10 may include one or more self-aligning switches 44. For example, a self-aligning switch 44 may be located on the control panel of the collimator box 15. As an additional example, a self-aligning switch 44 may be located on a side of the x-ray exposure hand switch 46.

The machine 10 may include one or more display screens 50. A display screen 50 may include a seven-segment display, a liquid crystal display, one or more light-emitting diodes (LED), a combination thereof, or any other suitable display. One or more display screens 50 may be located on the control panel of the collimator box 15.

When the portable x-ray machine 10 is powered on, the MCU 19 may receive power and begin a start-up sequence. The controller software of the MCU 19 may configure, calibrate, and/or zero both accelerometers 18, 22 and both stepper motors 25, 34 at start-up. The cassette accelerometer 18 may begin sending inclination data, orientation data, or the like to the MCU 19 in real time. The collimator box accelerometer 22 may begin sending inclination data, orientation data, or the like to the MCU 19 in real time. The MCU 19 may receive the data from the accelerometers 18, 22 simultaneously and in real time. The MCU 19 may output the X-axis and Y-axis inclination of one or both accelerometers 18, 22 to a display screen 50. The MCU 19 may send the inclination data from both accelerometers 18, 22 to onboard computer of the x-ray machine 10. The onboard computer may, in turn, display the inclination data.

To use the x-ray machine 10, an x-ray technologist may select an exam protocol through the onboard computer's input console. The exam protocol data may be sent to the MCU 19, which may calculate the necessary inclination for the X-axis and Y-axis of the x-ray collimator box 15 relative to the digital x-ray cassette 16 for the exam protocol. The MCU 19 may continue to monitor the X-axis and Y-axis of the x-ray collimator box 15 which it continues to do as the inclination of the collimator 15 changes until it is properly positioned. For best results, the collimator box 15 may first be manually positioned in the general direction of the x-ray cassette 16 before pressing the self-aligning switch 44. This may allow the MCU 19 to sense which direction the x-ray collimator box 15 is facing via the data from the collimator accelerometer 22 and adjust the inclination of the stepper motors 26, 34 appropriately towards the correct direction according to the exam protocol. Once a self-aligning switch 44 is pressed, the MCU 19 may activate the X-axis stepper motor 26 and/or the Y-axis stepper motor 34 to move to their corresponding inclination position according to the inclination of the digital x-ray cassettes 16 inclination and the selected exam protocol. The switch 44 may act like a deadman switch and need to be pressed until the collimator box 15 is in position. Once all the elements of the x-ray machine 10 are in position, the x-ray exposure may be activated by an x-ray technologist. After the exposure is taken, the inclination data of both the digital cassette 16 and the collimator box 15 may be recorded along with the digital x-ray image.

The inclination data may, for example, appear in the digital x-ray image, or it may be recorded as metadata in the image file, thereby allowing the inclination data to be used by a radiologist or a physician in making a diagnosis based on the x-ray image. Specifically, the radiologist can give a precise and accurate dictation of the positioning of the patient via the inclination data recorded from the system on the digital x-ray image, thus allowing the radiologist or physician to make a diagnosis that is fully-informed by the inclination data. For diagnostic purposes, this is a great improvement over the current practices of radiologists who use any of the other portable x-ray units for diagnostic analysis because none of the existing portable x-ray units provide the accuracy and certainty in knowing the precise measurements of the inclination data.

Persons of ordinary skill in the art may appreciate that numerous design configurations may be possible to enjoy the functional benefits of the inventive systems. Thus, given the wide variety of configurations and arrangements of embodiments of the present invention the scope of the present invention is reflected by the breadth of the claims below rather than narrowed by the embodiments described above.

What is claimed is:

1. A system for robotically self-aligning components of an x-ray machine to record inclination data and produce an accurate digital x-ray image of a patient to allow a radiologist to precisely and accurately dictate positioning of the patient via the inclination data recorded on the digital x-ray image, the system comprising:
a first accelerometer disposed in a digital cassette of an x-ray machine and configured to determine an inclination of the digital cassette on at least two axes and to transmit digital cassette inclination data associated with the determined digital cassette inclination;
a second accelerometer disposed in a collimator box of the x-ray machine and configured to determine an inclination of the collimator box on at least two axes and to transmit collimator box inclination data associated with the determined collimator box inclination;
a micro control unit (MCU) connected to the first accelerometer and to the second accelerometer, the MCU configured to receive the inclination data from the first accelerometer and from the second accelerometer;
a first stepper motor disposed in a first joint of the x-ray machine, the first stepper motor connected to the MCU and configured to adjust the angle, along a first axis, of the collimator box relative to the digital cassette based on instructions received from the MCU; and
a second stepper motor disposed in a second joint of the x-ray machine, the second stepper motor connected to the MCU and configured to adjust the angle, along a second axis, of the collimator box relative to the digital cassette based on instructions received from the MCU.

2. The system of claim 1 further comprising:
a first power and communication cable that connects the first stepper motor to the MCU;
a second power and communication cable that connects the second stepper motor to the MCU; and
a third power and communication cable that connects the first accelerometer to the MCU.

3. The system of claim 1, wherein the first stepper motor is an X-axis stepper motor that is configured to adjust the angle of the collimator box along the X-axis and the second stepper motor is a Y-axis stepper motor that is configured to adjust the angle of the collimator box along the Y-axis.

4. The system of claim 1 further comprising a display screen that is configured to display digital cassette inclination data determined by the first accelerometer and collimator box inclination data determined by the second accelerometer.

5. The system of claim 1 further comprising a self-aligning switch configured to activate, by way of the MCU, either of the first stepper motor and the second stepper motor based on the determined inclination of the digital cassette.

6. The system of claim 5, wherein either of the first stepper motor and the second stepper motor is further activated by the self-aligning switch based on a selected exam protocol.

7. A robotically assisted self-alignment method for aligning a set of components of a portable x-ray machine according to a patient exam protocol in order to produce an x-ray image with inclination data that allows a medical professional to accurately and precisely dictate a position of the patient for the x-ray image, said robotically assisted self-alignment method comprising:
configuring a set of accelerometers and a set of stepper motors;
receiving inclination data for a digital cassette and a collimator box from the set of accelerometers;
transmitting X-axis and Y-axis inclination data to a main control unit;
receiving selection of a patient exam protocol;
engaging a self-alignment control switch; and
adjusting the inclination of the collimator box according to the inclination of the digital cassette.

8. The robotically assisted self-alignment method of claim 7, wherein adjusting the inclination of the collimator box according to the inclination of the digital cassette exposes the digital cassette to x-rays activated through the collimator box.

9. The robotically assisted self-alignment method of claim 7, wherein the set of accelerometers comprises a collimator box accelerometer disposed within the collimator box and a cassette accelerometer disposed within the digital cassette.

10. The robotically assisted self-alignment method of claim 7, wherein the set of stepper motors comprises an X-axis stepper motor and a Y-axis stepper motor.

11. The robotically assisted self-alignment method of claim 10, wherein adjusting the inclination of the collimator box according to the inclination of the digital cassette comprises at least one of activating an X-axis stepper motor to adjust an angle of the collimator box along the X-axis and activating a Y-axis stepper motor to adjust an angle of the collimator box along the Y-axis.
